(19) United States
(12) Patent Application Publication
(10) Pub. No.: US 2026/0224884 A1
(43) Pub. Date: Aug. 6, 2026

(54) BLOOD PUMP AND METHOD OF MANUFACTURING A BLOOD PUMP HOUSING

(71) Applicant: ABIOMED Europe GmbH, Aachen (DE)

(72) Inventors: Frank Kirchhoff, Aachen (DE); Werner Carell, Aachen (DE)

(21) Appl. No.: 19/155,617

(22) PCT Filed: Feb. 13, 2024
(86) PCT No.: PCT/EP2024/053536

§ 371 Date: Aug. 12, 2025

(30) Foreign Application Priority Data
Feb. 15, 2023 (EP) 23156892.4

(51) Int. Cl. A61M 60/81; A61M 60/165; A61M 60/216; A61M 60/416; A61M 60/818; A61M 60/829

(52) U.S. Cl. CPC A61M 60/81; A61M 60/165; A61M 60/216; A61M 60/416; A61M 60/818; A61M 60/829; A61M 2207/00

(57) ABSTRACT

The present invention relates to a blood pump (10) comprising a blood pump housing (12), a shaft (14) and a pump element (16). The blood pump housing (12) comprises a first housing part (18) having a distal end (20) and a proximal end (22). The first housing part (18) comprises at least one first blood flow opening (24) at the distal end (20), at least one second blood flow opening (26) disposed between the distal end (20) and the proximal end (22) and a shaft supporting portion (28). The first housing part (18) is integrally formed as a one-piece unitary member, wherein the shaft (14) is supported in the shaft supporting portion (28). The pump element (16) is supported on the shaft (14) so that the pump element (16) is at least partially disposed within the first housing part (18). The present invention further relates to a method of manufacturing a blood pump housing (12).

The present invention relates to a blood pump and to a method of manufacturing a blood pump housing.

### BACKGROUND OF THE INVENTION

Different types of blood pumps are known, such as axial blood pumps, centrifugal (i.e. radial) blood pumps or combinations of both possibilities, where the blood flow is caused by both radial and axial forces. Such blood pumps are intended to support a patient's heart and are generally inserted into the patient's heart via a blood vessel such as the aorta or femoral artery by means of a catheter through a vascular access in the patient's skin, i.e. percutaneously.

Such blood pumps typically comprise a blood pump housing which may be composed of multiple parts to facilitate assembly of the blood pump. A first housing part is provided to support the pump element e.g., an impeller having blades on its outer circumference. Therefore, the first housing part comprises at least one first blood flow opening at a distal end of the first housing part and at least one second blood flow opening disposed remote from the distal end of the first housing part. The at least one second blood flow opening may be disposed between the distal end of the first housing part and a proximal end of the first housing part.

The pump element is driven by a drive unit and causes a blood flow between the at least one first blood flow opening and the at least one second blood flow opening. The blood pump may be provided for left ventricular support so that a blood flow is generated from the first blood flow opening to the second blood flow opening. In this case, the first blood flow opening is a blood flow inlet and the second blood flow opening is a blood flow outlet. Such a blood pump is also called an intravascular blood pump. Further, blood pumps which are placed in a patient's heart may also be referred to as intracardiac blood pumps. Of course, such blood pumps may also be used as a right ventricular support and the blood pumps do not necessarily need to be deployed within the patient's heart or blood vessels. Rather, such blood pumps may also be deployed externally of the heart to establish a blood flow from the heart to the blood vessel or vice versa.

To establish the blood flow, the pump element is at least partially disposed within the first housing part and is generally supported on a shaft or axle, which is driven by the drive unit so that the pump element rotates at a chosen speed to generate the blood flow. To ensure that the blood pump functions properly, it is essential that the shaft runs as centered as possible. Off-center running of the shaft leads to increased wear and consequently to an impairment of the pump performance and, in the worst case, even to total failure of the blood pump.

Therefore, the shaft is usually supported at at least two shaft supporting portions, which are sufficiently spaced from each other. One of said shaft supporting portions is provided in close proximity to the pump element, with only a little gap between the pump element and the shaft supporting portion to allow for sufficient purging. Generally, said shaft supporting portion is provided as a sliding bearing which is disposed within the first housing part. During assembly of the blood pump, the sliding bearing is mounted to the first housing part and fixed in its position e.g., by gluing or application of an adhesive. Thereafter, the shaft is mounted to reach through the sliding bearing with the pump element being supported on the shaft.

During assembly, the sliding bearing may not be optimally and centrically aligned, which may result in off-center running of the shaft during operation of the blood pump. Furthermore, in case the blood pump housing is provided as a multi-part housing, attachment and fixation of an additional housing part to the first housing part may cause warping of the sliding bearing, for instance caused by a welding procedure to mount the additional housing part.

It is thus an object of the invention to improve the running of the shaft. It is a further object of the invention to provide a method of manufacturing a blood pump housing allowing for an improved running of the shaft.

### SUMMARY OF THE INVENTION

According to a first aspect, a blood pump comprises a blood pump housing, a shaft and a pump element. The blood pump housing comprises a first housing part. The first housing part has a distal end and a proximal end. The first housing part comprises at least one first blood flow opening at the distal end of the first housing part, at least one second blood flow opening disposed between the distal end of the first housing part and the proximal end of the first housing part and a shaft supporting portion. The first housing part is integrally formed as a one-piece unitary member. The shaft is supported in the shaft supporting portion and the pump element is supported on the shaft so that the pump element is at least partially disposed within the first housing part.

In other words, the shaft supporting portion is integrally formed as a member of the first housing part and not as a separate component which is mounted to the first housing part during assembly of the blood pump. A non-centrical alignment of the shaft supporting portion is thus inhibited. Furthermore, the risk of warping due to an attachment of an additional housing part is also greatly reduced, as the shaft supporting portion is integrally formed with the remainders of the first housing part so that its position within the housing part is fixed.

Preferably, the first housing part is made of a ferritic iron-chromium-aluminum alloy i.e., of a FeCrAl alloy. The ferritic iron-chromium-aluminum alloy may comprise between 1% and 7% of aluminum (Al). The ferritic iron-chromium-aluminum alloy may comprise between 20% and 25% of chromium (Cr). Preferably, the ferritic iron-chromium-aluminum alloy comprises 5.8% of Al and/or preferably between 20.5% and 23.5% of Cr. Further, the ferritic iron-chromium-aluminum alloy preferably comprises between 0% and 0.08% of carbon (C), between 0% and 0.7% of silicon (Si) and/or between 0% and 0.4% of Manganese (Mn). This allows for a superior form stability. Further, said material has superior oxidation properties and has a low tendency to ageing and a low resistance change. The ferritic iron-chromium-aluminum alloy may be Kanthal APM.

Preferably, the shaft supporting portion is disposed between the at least one second blood flow opening and the proximal end of the first housing part. This allows for an optimal support of the shaft.

Preferably, a shaft bearing surface is formed on the shaft supporting portion. The shaft bearing surface may comprise a ceramic material, in particular Al₂O₃. The shaft is supported at the shaft bearing surface, which thus acts like a sliding bearing. A ceramic material, and in particular Al₂O₃, has superior characteristics in terms of wear and corrosion resistance. The shaft bearing surface preferably has a thickness of 0.5 μm to 5 μm, preferably of 2 μm. The shaft supporting portion may be an annular shaft supporting portion with a central opening and the shaft bearing surface may be formed on the inner circumferential surface of the central opening.

Preferably, the shaft supporting portion comprises a first surface and a second surface. The first surface faces towards the distal end of the first housing part and the second surface faces towards the proximal end of the first housing part. The second surface may comprise at least one purge fluid guiding channel. The at least one purge fluid guiding channel preferably extends from the central opening in the radial direction. The at least one purge fluid guiding channel may have a curved shape. Thus, incoming purge fluid can be channeled and directed to the central opening and the shaft bearing surface respectively. This greatly inhibits accumulation of particles.

The second surface may comprise a circumferential groove disposed radially outwardly from the central opening. The circumferential groove preferably is concentric with the central opening and may be partially or completely circumferential. The circumferential groove is configured to accommodate an adhesive so that further components of the blood pump can be securely fixed to the fist housing part during assembly of the blood pump.

The blood pump housing of the blood pump may comprise a second housing part, wherein the second housing part may be attached to the proximal end of the first housing part. The second housing part is preferably made of a ferritic iron-chromium-aluminum alloy, wherein the ferritic iron-chromium-aluminum alloy preferably comprises between 1% and 7% of aluminum (Al) and/or between 20% and 25% of chromium (Cr), wherein the ferritic iron-chromium-aluminum alloy preferably comprises 5.8% of aluminum (Al) and/or between 20.5% and 23.5% of chromium (Cr). The second housing part is preferably made of the same material as the first housing part.

The second housing part may be configured to accommodate further components of the blood pump. In particular, the second housing part is preferably configured to accommodate the drive unit. The second housing part is preferably fixed to the first housing part via an adhesive.

Preferably, the shaft comprises a ceramic material. Preferably, the shaft is made from a ceramic material. Alternatively, the shaft may be made from a metallic material coated with the ceramic material. The ceramic material may be silicon carbide (SIC), aluminum toughened zirconia (ATZ), zirconia toughened aluminum (ZTA) or aluminum oxide (Al₂O₃).

According to a second aspect, a method of manufacturing a blood pump housing comprises the following steps: providing a first housing part as an integrally formed one-piece unitary member, the first housing part having a distal end and a proximal end, wherein the first housing part comprises at least one first blood flow opening at the distal end of the first housing part, at least one second blood flow opening disposed between the distal end of the first housing part and the proximal end of the first housing part and a shaft supporting portion; and forming a shaft bearing surface comprising a ceramic material on the shaft supporting portion. Preferably, the ceramic material comprises Al₂O₃.

Preferably, the first housing part is provided by subtractive machining and subsequent abrasive flow machining. Preferably, the first housing part is provided by turning a round steel and subsequent milling. Thereafter, the first housing part is subject to abrasive flow machining. This allows for a particularly high form stability and a low surface roughness. Thus, adhesion of particles, like blood particles, is greatly inhibited.

It has to be mentioned that abrasive flow machining is sometimes also called flow grinding, abrasive flow deburring or extrude honing. Said process is advantageous when used for finishing workpieces with complex shaped external and internal contours, like the first housing part.

Preferably, the step of forming the shaft bearing surface comprises tempering the first housing part. The first housing part may be tempered at 800° C. to 1300° C. Preferably, the first housing part is tempered in a heat treatment oven, wherein the first housing part is placed within the heat treatment oven before the heat treatment oven is heated. Due to the tempering, the surface of the first housing part oxidizes so that the ceramic material is formed. Preferably, the so formed shaft bearing surface has a thickness of 0.5 μm to 5 μm, preferably of 2 μm.

Preferably, the first housing part is made from a ferritic iron-chromium-aluminum alloy, wherein the ferritic iron-chromium-aluminum alloy preferably comprises between 1% and 7% of aluminum (Al). Preferably, the ferritic iron-chromium-aluminum alloy comprises between 20% and 25% of chromium (Cr). Preferably, the ferritic iron-chromium-aluminum alloy comprises 5.8% of Al and/or preferably between 20.5% and 23.5% of Cr. Further, the ferritic iron-chromium-aluminum alloy preferably comprises between 0% and 0.08% of carbon (C), between 0% and 0.7% of silicon (Si) and/or between 0% and 0.4% of Manganese (Mn). This allows for a superior form stability. Further, said material has superior oxidation properties and has a low tendency to ageing and a low resistance change. The ferritic iron-chromium-aluminum alloy may be Kanthal APM.

Preferably, the shaft supporting portion comprises a central opening, wherein the central opening is honed prior to tempering, and wherein the shaft bearing surface is formed on the inner circumferential surface of the central opening.

### BRIEF DESCRIPTION OF THE DRAWINGS

The foregoing summary as well as the following detailed description of preferred embodiments will be better understood when read in conjunction with the appended drawings. For the purpose of illustrating the present disclosure, reference is made to the drawings. However, the scope of the disclosure is not limited to the specific embodiments disclosed in the drawings.

In the drawings:

FIG. 1 is a side view of a blood pump comprising a first housing part according to a first embodiment;

FIG. 2 is a front view of the blood pump of FIG. 1;

FIG. 3 is a cross section along the line A-A shown in FIG. 2;

FIG. 4 is a side view of the first housing part according to the first embodiment;

FIG. 5 is a cross section along the line B-B shown in FIG. 4;

FIG. 6 is a view of a proximal end of the first housing part according to the first embodiment;

FIG. 7 is a view of a distal end of the first housing part according to the first embodiment;

FIG. 8 is a side view of a first housing part according to a second embodiment;

FIG. 9 is a cross section along the line C-C shown in FIG. 8;

FIG. 10 is a view of a proximal end of the first housing part according to the second embodiment; and

FIG. 11 is a view of a distal end of the first housing part according to the second embodiment.

### DETAILED DESCRIPTION

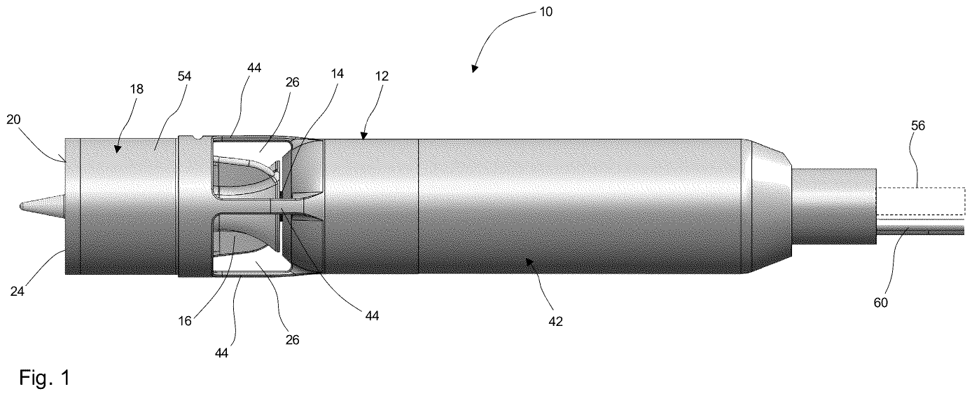

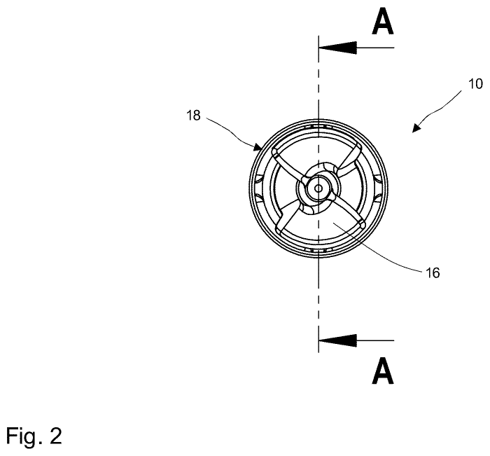

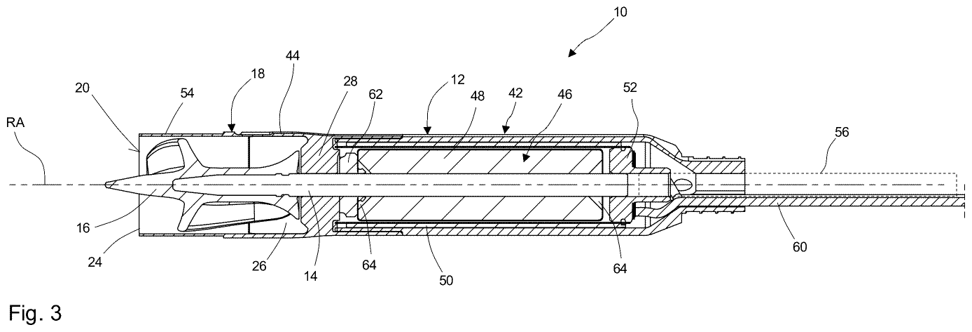

FIG. 1 depicts a side view of a blood pump 10 and FIG. 2 depicts a front view of the blood pump 10. In this exemplary embodiment, the blood pump 10 is an intravascular blood pump for left ventricular support. The blood pump 10 comprises a tubular-shaped blood pump housing 12, a shaft 14, a pump element 16, a drive unit 46 and a purge fluid line 56. The purge fluid line 56 is only schematically indicated in FIGS. 1 and 3, but may be configured as shown in US 2021/0339003 A1. In this exemplary embodiment, the shaft is completely made of aluminum toughened zirconia.

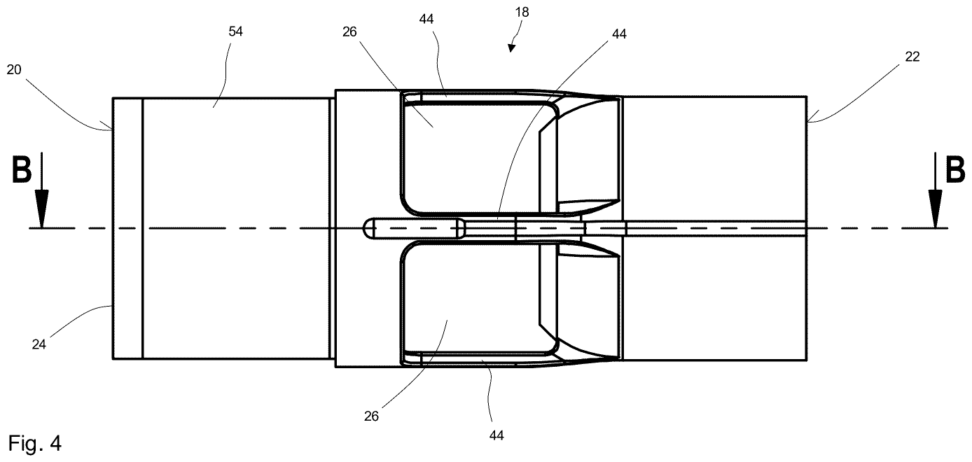

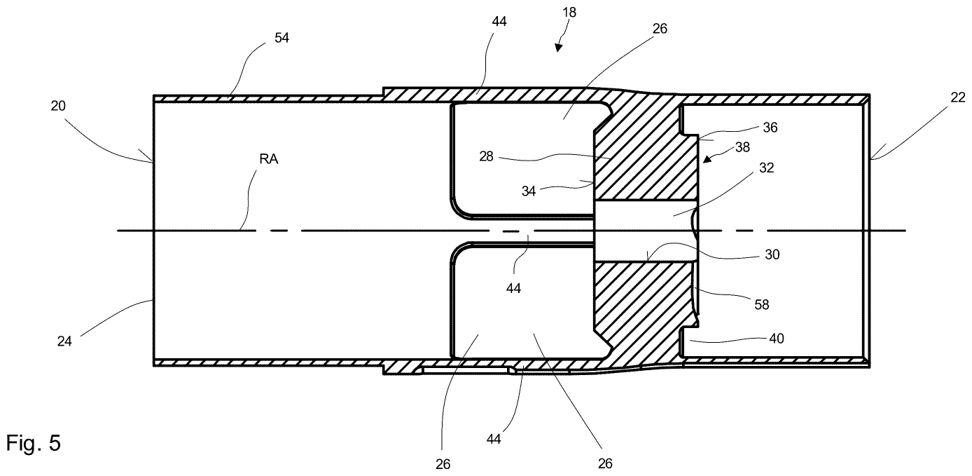

The blood pump housing 12 is a multi-part housing and comprises of a first housing part 18 and a second housing part 42. The first housing part 18 comprises an outer casing 54, a distal end 20 and a proximal end 22, see also FIGS. 4 and 5. A first blood flow opening 24 is provided at the distal end 20 of the first housing part 18. In addition, at least one second blood flow opening 26 is provided between the distal end 20 and the proximal end 22 of the first housing part 18. In this exemplary embodiment, the first housing part 18 comprises four second blood flow openings 26 which are provided equally spaced around the circumference of the first housing part 18 at the outer casing 54 and which form radial openings of the outer casing 54. The second blood flow openings 26 are separated by struts 44, in this exemplary embodiment by a total of four struts 44.

The pump element 16 is non-rotatably supported on the shaft 14, so that it is partially disposed within the first housing part 18. In this embodiment, the pump element 16 is an impeller having blades on its outer circumferential surface. The impeller 16 rotates together with the shaft 14 about a central axis of rotation RA, so that blood is conveyed between the first blood flow opening 24 and the plurality of second blood flow openings 26, see also FIG. 4. As the blood pump 10 in the shown embodiment is intended for left ventricular support, a blood flow is caused from the first blood flow opening 24 to the plurality of second blood flow openings 26 i.e., the first blood flow opening 24 is a blood flow inlet and each of the second blood flow openings 26 is a blood flow outlet.

Rotation of the shaft 14 is caused by the drive unit 46. As shown in FIG. 3, the drive unit 46 comprises a magnet 48 as a rotor and a stator coil 50. The magnet 48 is non-rotatably supported on the shaft 14 and is disposed radially within the stator coil 50. The stator coil 50 is housed within the blood pump housing 12, in particular within the second housing part 42. In this embodiment, the stator coil 50 is a multi-layer stator coil, preferably with two layers of windings embedded in a suitable synthetic material or synthetic resin. The magnet 48 comprises recessed portions 64 on both axial ends which are recessed towards the other one of the axial ends. During assembly of the blood pump 10, the recessed portions 64 are filed with an adhesive, as will be explained below in more detail. Energizing the stator coil 50 via a wiring 60 causes an alternating magnetic field which induces a force on the magnet 48 so that it rotates together with the shaft 14 about the central axis of rotation RA and, hence, with the impeller 16 to cause the blood flow as described above.

As can be seen in the cross section of FIG. 3, the shaft 14 is supported at two spaced apart points, namely at a shaft supporting portion 28 in close proximity to the impeller 16 and at a bearing washer 52 disposed axially behind the drive unit 46 in the second housing part 42 when viewed from the distal end 20 of the first housing part 18 to the proximal end 22 of the first housing part 18. Thus, the drive unit 42 is sandwiched between the shaft supporting portion 28 and the bearing washer 52. The bearing washer 52 may be a sliding bearing.

Furthermore, a washer 62 is supported on the shaft 14 between the shaft supporting portion 28 and the magnet 48. The washer 62 is intended to correctly position the magnet 48 on the shaft 14. The washer 62 may be a thrust washer.

The shaft supporting portion 28 is an annular member and an integral part of the first housing part 18. In other words, the first housing part 18 is formed as a one-piece unitary member comprising the outer casing 54 and the shaft supporting portion 28. The shaft supporting portion 28 is disposed radially inwardly of the outer casing 54 between the second blood flow openings 26 and the proximal end 22 of the first housing part 18. The shaft supporting portion 28 has a first surface 34 facing towards the distal end 20 of the first housing part 18 and a second surface 36 facing towards the proximal end 22 of the first housing part 18. The shaft supporting portion 28 comprises a central opening 32 extending between the first surface 34 and the second surface 36. The central opening 32 is concentric with the central axis of rotation RA and it comprises a shaft bearing surface 30 on its inner circumferential surface. The shaft bearing surface 32 covers the entire inner circumferential surface of the central opening 32 and comprises a ceramic material. The ceramic material may partially or completely be composed of Al₂O₃. In this exemplary embodiment, the shaft bearing surface 30 has a uniform thickness (i.e. an extension in the radial direction relative to the central axis of rotation RA) of less than 5 μm, preferably of about 2 μm. The formation of the shaft bearing surface 30 will be explained in more detail below.

As can be seen in FIG. 6, the second surface 36 of the shaft supporting portion 28 comprises a purge fluid guiding channel 38 and a circumferential groove 40. The purge fluid guiding channel 38 is recessed towards the first surface 34 of the shaft supporting portion 28. In this exemplary embodiment, the purge fluid guiding channel 38 comprises three sub-channels 58 which extend radially outwardly from the central opening 32 of the shaft supporting portion 28. As depicted in FIG. 6, the sub-channels 58 are evenly distributed around the central axis of rotation RA and each have an arc-shape. The circumferential groove 40 is also recessed towards the first surface 34 of the shaft supporting portion 28 and is located radially outwardly of the purge fluid guiding channel 38 at a transition portion between the shaft supporting portion 28 and the outer casing 54 of the first housing part 18. During assembly of the blood pump 10, the circumferential groove 40 is filed with an adhesive to fix the stator coil 50 to the blood pump housing 12, as will be explained below in more detail.

To keep blood from entering the central opening 32, the drive unit 46 and the bearing washer 52, a purge fluid is deployed via the purge fluid line 56, as commonly known. Further, the deployed purge fluid is used as a lubricant for the shaft bearing surface 30 and the bearing washer 52 supporting the shaft 14. The purge fluid enters the blood pump 10 at the bearing washer 52 with a pressure higher than the blood pressure present in order to prevent blood from penetrating into the blood pump housing 12 beyond the second blood flow openings 26. The purge fluid flows through the bearing washer 52 towards the shaft supporting portion 28. There, the purge fluid is guided through the sub-channels 58 of the purge fluid guiding channel 38 to the central opening 32 and therethrough to the back of the impeller 16 facing the first surface 34.

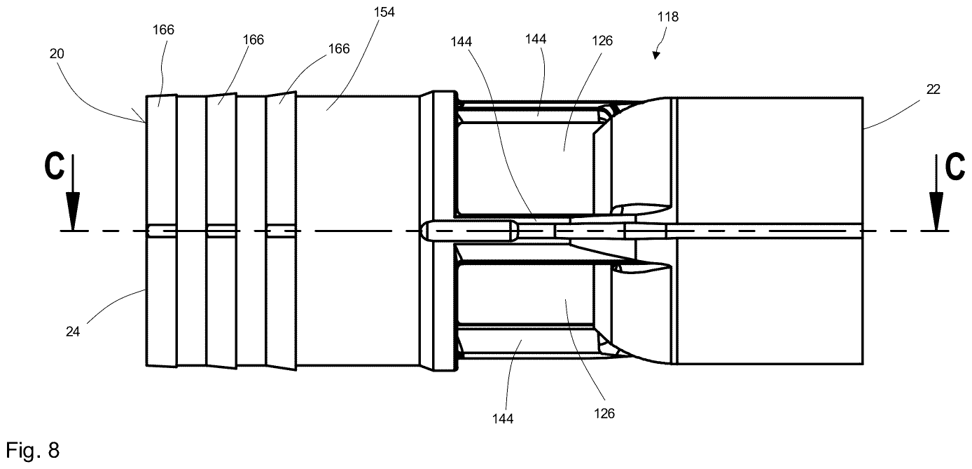

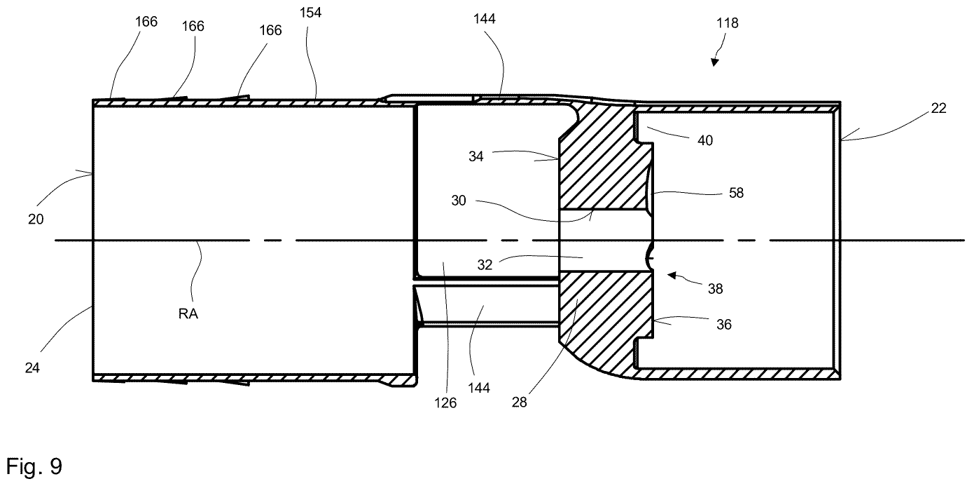

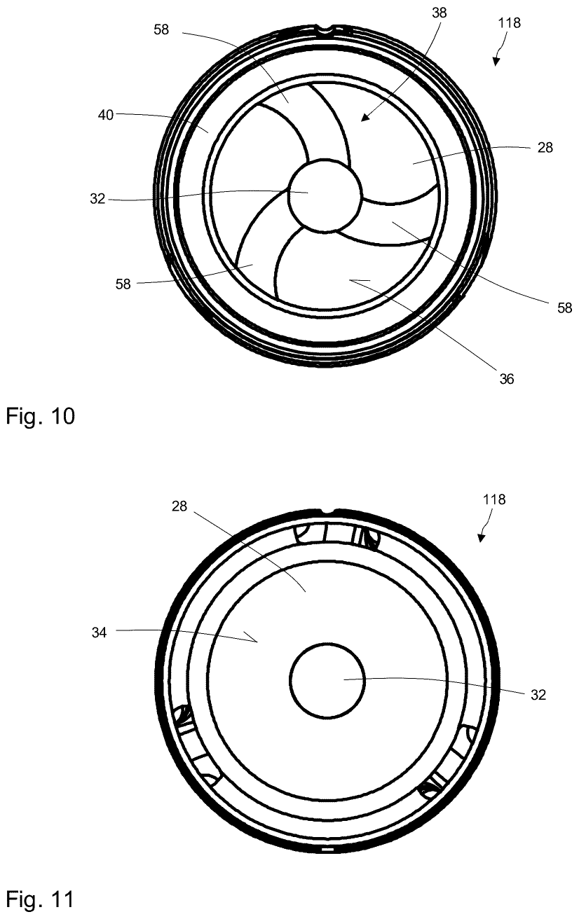

FIGS. 8 to 11 depict different views of a first housing part 118 according to a second embodiment. The first housing part 118 according to the second embodiment differs from the first housing part 18 according to the first embodiment in the configuration of the second blood flow openings 126 and the provision of mounting lugs 166.

According to the second embodiment, only three second blood flow openings 126 are provided, which are evenly distributed at the outer circumference of the first housing part 118 as radial openings in the outer casing 154. Accordingly, each second blood flow opening 126 is separated from an adjacent blood flow opening 126 by a strut 144, so that three struts 144 are provided in total. The total opening area of the second blood flow openings 126 of the first housing part 118 according to the second embodiment is larger than the opening area of the second blood flow openings 26 of the first housing part 18 according to the first embodiment. This is advantageous in terms of an unobstructed blood flow, but on the other hand slightly increases the manufacturing effort.

Further, mounting lugs 166 are provided on an outer circumferential surface of the first housing part 118 between the distal end 20 and the second blood flow openings 126. In this exemplary embodiment, a total of three mounting lugs 166 is provided. The mounting lugs 166 extend nearly completely around the outer circumference of the outer casing 154 of the first housing part 118 and are tapered in direction of the distal end 20 of the first housing part 118. The mounting lugs 116 offer a barb-like effect for the attachment of e.g., a suction hose or a cannula.

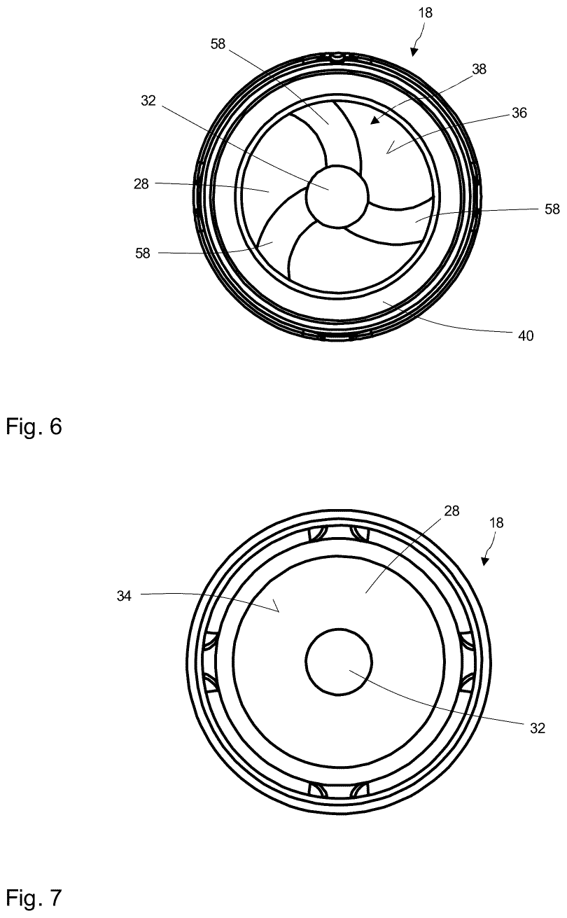

Next, a method of manufacturing the blood pump housing 12 and the steps for assembly of the blood pump 10 are described for the first embodiment of the blood pump 10 shown in FIGS. 1 to 7. Of course, the description applies equally to the first housing part 118 according to the second embodiment shown in FIGS. 8 to 11.

The first housing part 18 is provided by machining a round steel. The round steel is a ferritic iron-chromium-aluminum alloy, wherein the ferritic iron-chromium-aluminum alloy preferably comprises between 1% and 7% of aluminum (Al) and between 20% and 25% of chromium (Cr). Preferably, the ferritic iron-chromium-aluminum alloy comprises 5.8% of aluminum (Al) and between 20.5% and 23.5% of chromium (Cr). Further, the ferritic iron-chromium-aluminum alloy preferably comprises between 0% and 0.08% of carbon (C), between 0% and 0.7% of silicon (Si) and between 0% and 0.4% of Manganese (Mn). The ferritic iron-chromium-aluminum alloy may be Kanthal APM.

The round steel is machined by subtractive machining and subsequent abrasive flow machining to achieve superior surface conditions. Next, the so-formed central opening 32 of the shaft supporting portion 28 is honed to achieve a diameter accuracy of ±0.1 μm. After honing, the first housing part 18 is tempered at a temperature of 800° C. to 1300° C. The oven used for tempering is not pre-heated, but the first housing part 18 is placed into the oven which is subsequently heated to the intended temperature. Likewise, the first housing part 18 is not quenched or removed from the oven after tempering, but left inside the oven to cool down. Said tempering step forms a ceramic layer comprising of Al₂O₃ of even thickness on the outer surface of the first housing part 18. In particular, the shaft bearing surface 30 is formed during the tempering step with a thickness of about 2 μm. The second housing part 42 is provided accordingly, i.e. by machining a round steel of the identical material with subsequent tempering as described for the first housing part 18.

To assemble the blood pump 10, the shaft 14 with the already attached impeller 16 is slid through the first blood flow opening 24 into the first housing part 18. In particular, the shaft 14 is slid through the central opening 32 until only a small gap remains between the impeller 16 and the first surface 34 of the shaft supporting portion 28. A spacer may be introduced through one or more of the second blood flow openings 26 to warrant a correct position of the impeller 16 and the shaft 14 relative to the first housing part 18.

Next, the circumferential groove 40 is filed with a ring composed of an adhesive and the washer 62 is slid over the shaft 14 until it abuts the second surface 36 of the shaft supporting portion 28. Thereafter, the magnet 48 is installed, wherein the recessed portion 64 facing the washer 62 is filed with an adhesive. For instance, said adhesive may be provided as a ring composed of adhesive slid over the shaft 14 prior to installation of the magnet 48. Thereafter, the recessed portion 64 on the other axial end of the magnet is filed with an adhesive. The adhesives secure the magnet 48 to the shaft 14 and further inhibit intrusion of purge fluid during operation of the blood pump 10, which might otherwise cause corrosion.

Next, a preassembled element consisting of the stator coil 50 and the bearing washer 52 is slid onto the shaft 14 to the correct position where it abuts the adhesive supported in the circumferential groove 40. The so far applied adhesives are then cured by a process depending on the type of adhesive used e.g., by applying heat. The purge fluid line 56 is thereafter glued to the bearing washer 52.

Then the second housing part 42 is attached to the proximal end 22 of the first housing part 18. In particular, an adhesive is applied between the overlapping area consisting of a part of the outer circumferential surface of the second housing part 42 and a part of the inner circumferential surface of the first housing part 18. In addition, an adhesive is also applied between the second housing part 42 and the purge fluid line 56. The so far applied adhesives are then cured by a process depending on the type of adhesive used e.g., by applying heat.

### EXEMPLARY IMPLEMENTATIONS

As already described, the technology described herein may be implemented in various ways. In that regard, the foregoing disclosure is intended to include, but not be limited to, the systems, methods, and combinations and sub-combinations thereof that are set forth in the following exemplary implementations. Preferred embodiments are described in the following paragraphs:
A1 Blood pump comprising a blood pump housing, a shaft and a pump element, wherein the blood pump housing comprises a first housing part, the first housing part having a distal end and a proximal end, wherein the first housing part comprises at least one first blood flow opening at the distal end of the first housing part, at least one second blood flow opening disposed between the distal end of the first housing part and the proximal end of the first housing part and a shaft supporting portion, wherein the first housing part is integrally formed as a one-piece unitary member, wherein the shaft is supported in the shaft supporting portion, and wherein the pump element is supported on the shaft so that the pump element is at least partially disposed within the first housing part.
A2 Blood pump according to paragraph A1, wherein the first housing part is made of a ferritic iron-chromium-aluminum alloy.
A3 Blood pump according to paragraph A2, wherein the ferritic iron-chromium-aluminum alloy comprises between 1% and 7% of aluminum and/or between 20% and 25% of chromium, wherein the ferritic iron-chromium-aluminum alloy preferably comprises 5.8% of aluminum and/or between 20.5% and 23.5% of chromium.
A4 Blood pump according to paragraph A2 or A3, wherein the ferritic iron-chromium-aluminum alloy comprises between 0% and 0.08% of carbon and/or between 0% and 0.7% of silicon and/or between 0% and 0.4% of Manganese.
A5 Blood pump according to any one of the preceding paragraphs A1 to A4, wherein the first housing part is made of Kanthal APM.
A6 Blood pump according to any one of the preceding paragraphs A1 to A5, wherein the shaft supporting portion is disposed between the at least one second blood flow opening and the proximal end of the first housing part.
A7 Blood pump according to any one of the preceding paragraphs A1 to A6, wherein a shaft bearing surface is formed on the shaft supporting portion.
A8 Blood pump according to paragraph A7, wherein the shaft bearing surface comprises a ceramic material.
A9 Blood pump according to paragraph A8, wherein the ceramic material is partially or completely composed of Al₂O₃.
A10 Blood pump according to any one of the preceding paragraphs A1 to A9, wherein the shaft supporting portion is an annular shaft supporting portion with a central opening.
A11 Blood pump according to paragraph A10, wherein the shaft bearing surface is formed on the inner circumferential surface of the central opening.
A12 Blood pump according to paragraph A10 or A11, wherein the central opening is concentric with a central axis of rotation of the shaft.
A13 Blood pump according to any one of the preceding paragraphs A1 to A12, wherein the shaft supporting portion comprises a first surface and a second surface, the first surface facing towards the distal end of the first housing part and the second surface facing towards the proximal end of the first housing part.
A14 Blood pump according to paragraph A13, wherein the second surface comprises at least one purge fluid guiding channel.
A15 Blood pump according to paragraph A13 or A14, wherein the second surface comprises a circumferential groove disposed radially outwardly from the central opening.
A16 Blood pump according to any one of the preceding paragraphs A1 to A15, wherein the blood pump housing comprises a second housing part.
A17 Blood pump according to paragraph A16, wherein the second housing part is attached to the proximal end of the first housing part, preferably by an adhesive.
A18 Blood pump according to paragraph A16 or A17, wherein the second housing part is made of a ferritic iron-chromium-aluminum alloy.
A19 Blood pump according to paragraph A18, wherein the ferritic iron-chromium-aluminum alloy comprises between 1% and 7% of aluminum and/or between 20% and 25% of chromium, wherein the ferritic iron-chromium-aluminum alloy preferably comprises 5.8% of aluminum and/or between 20.5% and 23.5% of chromium.
A20 Blood pump according to paragraph A18 or A19, wherein the ferritic iron-chromium-aluminum alloy comprises between 0% and 0.08% of carbon and/or between 0% and 0.7% of silicon and/or between 0% and 0.4% of Manganese.
A21 Blood pump according to any one of the preceding paragraphs A18 to A20, wherein the second housing part is made of Kanthal APM.
A22 Blood pump according to any one the preceding paragraphs A1 to A21, further comprising a drive unit configured to drive the shaft.
A23 Blood pump according to paragraph A22, wherein the drive unit comprises a stator coil and a magnet.
A24 Blood pump according to any one of the preceding paragraphs A1 to A23, wherein the circumferential groove of the shaft supporting portion is at least partially filed with an adhesive.
A25 Blood pump according to paragraph A24, wherein the stator coil is fixed to the first housing part by the adhesive provided in the circumferential groove.
A26 Blood pump according to any one of the preceding paragraphs A23 to A25, wherein the magnet is supported on the shaft, wherein the magnet is preferably rigidly fixed to the shaft.
A27 Blood pump according to paragraph A23 or A26, wherein the magnet comprises a first recessed portion at one axial end of the magnet.
A28 Blood pump according to paragraph A27, wherein the magnet comprises a second recessed portion at the other axial end of the magnet.
A29 Blood pump according to paragraph A27 or A28, wherein the first recessed portion and/or the second recessed portion is filed with an adhesive.
A30 Blood pump according to any one of the preceding paragraphs A1 or A29, wherein a washer is supported on the shaft.
A31 Blood pump according to paragraph A30, wherein the washer is abutting the shaft supporting portion.
A32 Blood pump according to paragraph A31, wherein the washer is abutting the second surface of the shaft supporting portion.
A32 Blood pump according to any one of the preceding paragraphs A30 to A32, wherein the washer is provided between the magnet and the shaft supporting portion.
A33 Blood pump according to any one of the preceding paragraphs A22 to A32, wherein the drive unit is disposed within the blood pump housing, preferably within the second housing part.
A34 Blood pump according to any one of the preceding paragraphs, wherein the blood pump comprises a bearing washer supporting the shaft.
A35 Blood pump according to paragraph A34, wherein the bearing washer is disposed within the blood pump housing.
A36 Blood pump according to paragraph A34 or A35, wherein the bearing washer is disposed within the second housing part.
A37 Blood pump according to any one of the preceding paragraphs A34 to A36, wherein the magnet is disposed between the bearing washer and the shaft supporting portion.
A38 Blood pump according to any one of the preceding paragraphs A34 to A37, wherein the magnet is disposed between the bearing washer and the washer.
A39 Blood pump according to any one of the preceding paragraphs A1 to A38, wherein at least one mounting lug is provided on an outer peripheral surface of the first housing part.
A40 Blood pump according to paragraph A39, wherein the at least one mounting lug at least partially extends circumferentially about the outer peripheral surface of the first housing part.
A41 Blood pump according to paragraph A39 or A40, wherein the at least one mounting lug is provided between the distal end of the first housing part and the at least one second blood flow opening.
A42 Blood pump according to any one of the preceding paragraphs A1 to A41, wherein the shaft comprises a ceramic material.
A43 Blood pump according to paragraph A42, wherein the shaft is made from the ceramic material.
A44 Blood pump according to paragraph A42, wherein the shaft is made from a metallic material coated with the ceramic material.
A45 Blood pump according to any one of the preceding paragraphs A42 to A44, wherein the ceramic material is silicon carbide or aluminum toughened zirconia or zirconia toughened aluminum or aluminum oxide.
A46 Blood pump according to any one of the preceding paragraphs A1 to A45, wherein the first housing part comprises an outer casing, wherein the outer casing is integrally formed with the shaft supporting portion.
B1 Method of manufacturing a blood pump housing, wherein the method comprises the following steps: providing a first housing part as an integrally formed one-piece unitary member, the first housing part having a distal end and a proximal end, wherein the first housing part comprises at least one first blood flow opening at the distal end of the first housing part, at least one second blood flow opening disposed between the distal end of the first housing part and the proximal end of the first housing part and a shaft supporting portion; and forming a shaft bearing surface comprising a ceramic material on the shaft supporting portion
B2 Method according to paragraph B1, wherein the ceramic material comprises Al₂O₃.
B3 Method according to paragraph B1 or B2, wherein the first housing part is provided by subtractive machining and subsequent abrasive flow machining.
B4 Method according to paragraph B3, wherein subtractive machining includes grinding and/or milling and/or honing and/or turning and/or electro-discharge machining.
B5 Method according any one of the preceding paragraphs B1 to B4, wherein the step of forming the shaft bearing surface comprises tempering the first housing part.
B6 Method according to paragraph B5, wherein the first housing part is tempered at 800° C. to 1300° C.
B7 Method according to paragraph B5 or B6, wherein first housing part is tempered in a heat treatment oven, wherein the first housing part is placed within the heat treatment oven before the heat treatment oven is heated.
B8 Method according to any one of the preceding paragraphs B1 to B7, wherein the first housing part is made from a ferritic iron-chromium-aluminum alloy.
B9 Method according to paragraph B8, wherein the ferritic iron-chromium-aluminum alloy comprises between 1% and 7% of aluminum and/or between 20% and 25% of chromium, wherein the ferritic iron-chromium-aluminum alloy preferably comprises 5.8% of aluminum and/or between 20.5% and 23.5% of chromium.
B10 Method according to paragraph B8 or B9, wherein the ferritic iron-chromium-aluminum alloy comprises between 0% and 0.08% of carbon and/or between 0% and 0.7% of silicon and/or between 0% and 0.4% of Manganese.
B11 Method according to any one of the preceding paragraphs B1 to B10, wherein the first housing part is made of Kanthal APM.
B12 Method according to any one of the preceding paragraphs B1 to B11, wherein the shaft supporting portion comprises a central opening, wherein the central opening is honed prior to tempering, and wherein the shaft bearing surface is formed on the inner circumferential surface of the central opening.
B13 Method according to any one of the preceding paragraphs B1 to B12, wherein the method comprises the step of providing a second housing part.
B14 Method according to paragraph B15, wherein the second housing part is tempered at 800° C. to 1300° C.
B15 Method according to paragraph B13 or B14, wherein second housing part is tempered in a heat treatment oven, wherein the second housing part is placed within the heat treatment oven before the heat treatment oven is heated.
B16 Method according to any one of the preceding paragraphs B1 to B15, wherein the second housing part is made is from a ferritic iron-chromium-aluminum alloy.
B17 Method according to paragraph B16, wherein the ferritic iron-chromium-aluminum alloy comprises between 1% and 7% of aluminum and/or between 20% and 25% of chromium, wherein the ferritic iron-chromium-aluminum alloy preferably comprises 5.8% of aluminum and/or between 20.5% and 23.5% of chromium.
B18 Method according to paragraph B16 or B17, wherein the ferritic iron-chromium-aluminum alloy comprises between 0% and 0.08% of carbon and/or between 0% and 0.7% of silicon and/or between 0% and 0.4% of Manganese.
B19 Method according to any one of the preceding paragraphs B1 to B18, wherein the second housing part is made of Kanthal APM.
B20 Method according to any one of the preceding paragraphs B1 to B19, wherein the second housing part is fixed to the first housing part by an adhesive.

As utilized herein, the terms “approximately”, “about”, “substantially” and similar terms are intended to have a broad meaning in harmony with the common and accepted usage by those of ordinary skill in the art to which the subject matter of this disclosure pertains. It should be understood by those of skill in the art who review this disclosure that these terms are intended to allow a description of certain features described without restricting the scope of these features to the precise numerical ranges provided. Accordingly, these terms should be interpreted as indicating that insubstantial or inconsequential modifications or alterations of the subject matter described and are considered to be within the scope of the disclosure. The terms “at least partially” or “partially” as used herein mean both partial and entirely or complete respectively.

Herein, “proximal” and “distal” are seen relative to the medical staff or physician. Thus, proximal designates something which is relatively close to the physician whereas distal designates something which is relatively far away from the physician when the blood pump is introduced into the patient's body. In addition, numerals like “first” or “second” do not denote a specific order, but are only intended to distinguish the respective elements and features from another.

### LIST OF REFERENCE SIGNS

10 blood pump
12 blood pump housing
14 shaft
16 pump element/impeller
18, 118 first housing part
20 distal end of first housing part
22 proximal end of first housing part
24 first blood flow opening
26, 126 second blood flow openings
28 shaft supporting portion
30 shaft bearing surface
32 central opening of shaft supporting portion
34 first surface of shaft supporting portion
36 second surface of shaft supporting portion
38 purge fluid guiding channel
40 circumferential groove
42 second housing part
44, 144 strut
46 drive unit
48 magnet
50 stator coil
52 bearing washer
54, 154 outer casing of first housing part
56 purge fluid line
58 sub-channel
60 wiring
62 washer
64 recessed portion of magnet
166 mounting lugs
RA central axis of rotation

1. A blood pump comprising:
a blood pump housing comprising a first housing part, the first housing part having a distal end and a proximal end, wherein the first housing part comprises at least one blood flow opening at the distal end of the first housing part, at least one second blood flow opening disposed between the distal end of the first housing part and the proximal end of the first housing part, and a shaft supporting portion, wherein the first housing part is integrally formed as a one-piece unitary member;
a shaft supported in the shaft supporting portion; and
a pump element, wherein the pump element is supported on the shaft so that the pump element is at least partially disposed within the first housing part.

2. The blood pump according to claim 1, wherein the first housing part comprises a ferritic iron-chromium-aluminum alloy.

3. The blood pump according to claim 2, wherein the ferritic iron-chromium-aluminum alloy comprises between 1% and 7% of aluminum (Al) and/or between 20% and 25% of chromium (Cr).

4. The blood pump according to claim 1, wherein the shaft supporting portion is disposed between the at least one second blood flow opening and the proximal end of the first housing part.

5. The blood pump according to claim 1, wherein the shaft supporting portion includes a shaft bearing surface, and wherein the shaft bearing surface comprises a ceramic material.

6. The blood pump according to claim 5, wherein the shaft supporting portion is an annular shaft supporting portion with a central opening, wherein the shaft bearing surface is formed on an inner circumferential surface of the central opening.

7. The blood pump according to claim 6, wherein the shaft supporting portion comprises a first surface and a second surface, the first surface facing towards the distal end of the first housing part and the second surface facing towards the proximal end of the first housing part, wherein the second surface comprises at least one purge fluid guiding channel and/or wherein the second surface comprises a circumferential groove disposed radially outwardly from the central opening.

8. The blood pump according to claim 1, wherein the blood pump housing comprises a second housing part, wherein the second housing part is attached to the proximal end of the first housing part.

9. The blood pump according to claim 1, wherein the shaft comprises a ceramic material.

10. A method of manufacturing a blood pump housing, wherein the method comprises:
providing a first housing part as an integrally formed one-piece unitary member, the first housing part having a distal end and a proximal end, wherein the first housing part comprises at least one first blood flow opening at the distal end of the first housing part, at least one second blood flow opening disposed between the distal of the first housing part and the proximal end of the first housing part and a shaft supporting portion; and
forming a shaft bearing surface comprising a ceramic material on the shaft supporting portion.

11. The method according to claim 10,
wherein the first housing part is provided by subtractive machining and subsequent abrasive flow machining.

12. The method according to claim 10, wherein forming the shaft bearing surface comprises tempering the first housing part.

13. The method according to claim 12, wherein the first housing part is tempered at 800° C. to 1300° C.

14. The method according to claim 10, wherein the first housing part is made from a ferritic iron-chromium-aluminum alloy.

15. The method according to claim 10, wherein the shaft supporting portion comprises a central opening, wherein the central opening is honed prior to tempering, and wherein the shaft bearing surface is formed on an inner circumferential surface of the central opening.
